(19) United States
(12) Patent Application Publication
(10) Pub. No.: US 2008/0021322 A1
(43) Pub. Date: Jan. 24, 2008

(54) Ultrasonic imaging apparatus and method

(76) Inventors: Michael Benjamin Stone, Oakland, CA (US); Samuel Rascher Brooks, Brooklyn, NY (US)

Correspondence Address: PATENT LAW PROFESSIONALS, P.O. BOX 612407, SAN JOSE, CA, 95161, US

(21) Appl. No.: 11/440,565

(22) Filed: May 24, 2006

(51) Int. Cl. A61B 8/00

(52) U.S. Cl. 600/443

(57) ABSTRACT

A hand-held ultrasonic imaging apparatus having an integrated display and transducer. The transducer and display are relatively configured so that an operator may look directly down at the patient and, at the same time visualize images of the patient's internal body structures on the display. The apparatus may be used to facilitate placement and insertion of a needle or catheter into a patient. Images displayed on the screen (e.g., the tip of the needle or catheter and an intended internal target body structure) and the actual needle or catheter itself may be maintained within the same visual axis of the operator, thereby avoiding any need to look away from the needle or catheter in order to visualize images on the display. The apparatus may also include an integrated transducer control and signal processing circuit, which together with the integrated display and transducer, provide a complete integrated ultrasonic imaging solution.

### BACKGROUND OF THE INVENTION

Medical ultrasonography is an ultrasonic-based imaging technique used to capture images of internal body structures (e.g., organs, bones, cartilage, and other anatomical tissues) of patients in real time. The ability to capture images in real time allows doctors to rapidly diagnose illnesses and perform time critical therapeutic procedures.

FIG. 1 illustrates the use of a typical prior art ultrasonic imaging system. The system comprises a hand-held probe 100, a computer 102, and a display 104. A first end of the probe 100 contains a phased array of piezoelectric transducers, which generate sound pulses in response to electrical signals received from the computer 102, via a cable 106. A doctor, clinician or other operator 108 moves the first end of the probe 100 (commonly referred to in the art as the “scan head”) over the patient's 110 skin, while sound pulses from the transducer are directed into the patient's 110 body. To facilitate movement and enhance coupling, a small amount of water-based gel is often applied between the scan head and the patient's body 110.

As the operator 108 moves the scan head across the skin of the patient 110, sound pulses from the transducer are reflected from the internal body structures of the patient 110. Some of the reflected sound pulses impinge on the piezoelectric material of the transducer, which causes the transducer to vibrate. The piezoelectric transducer is a special type of material which when vibrated converts sound waves into electrical signals characterizing the internal body structures from which the pulses were reflected. These electrical signals are electrically coupled to a second end of the probe 100 and transmitted, via the cable 106, to the computer 102, where they are digitized and processed to generate display signals for displaying images of the scanned internal body structures on the display 104.

A major drawback of conventional ultrasonic imaging systems, such as the one shown in FIG. 1, is that the probe operator 108 must look away from the patient 110 (as indicated by the arrow in FIG. 1) in order to view the display 104. The necessity of having to look away from the patient 110 in order to view the display 104 not only can be disorienting for the operator 108, it can also lead to patient injury, particularly when the system is used to assist in invasive procedures such as insertion of intravenous catheters or fluid drainage needles.

Another drawback of conventional ultrasonic imaging systems is that they are often bulky. This drawback limits their application and potential utility. In hospital settings, for example, hospital patient and examining rooms are often small, leaving little room for bulky equipment. Consequently, ultrasound imaging systems are typically dedicated to special examination rooms, where they are not as readily available as would be desired.

Attempts have been made to reduce the size of conventional ultrasonic imaging systems, so that the systems are portable. For example, U.S. Pat. No. 6,746,402 to Ustuner discloses an ultrasonic imaging system comprising a wearable ultrasonic probe in the form of a glove. The glove is worn on the operator's hand, and includes a transducer placed around the distal end of a finger. The glove may also include a display element (e.g., a liquid crystal display (LCD)) and/or a processor disposed on the dorsum of the glove, as shown in FIG. 5B of the Ustuner patent.

U.S. Pat. No. 5,817,024 to Ogle et al. and U.S. Pat. No. 6,569,102 to Imran et al. also disclose portable ultrasonic imaging apparatuses. Both apparatuses are configured within a hand-held housing. The housings have spaced-apart parallel front and rear walls, spaced-apart generally parallel side walls, a top wall, and a side wall. The bottom wall includes a transducer having a scan surface that is substantially perpendicular to the front, rear and side walls. The front wall is provided with a display screen and a plurality of control buttons. As explained in more detail below, the portable ultrasonic imaging apparatus in the Imran et al. patent may be inserted in a probe guide mechanism that has a semicircular recess adapted to receive and guide the insertion of a hypodermic needle.

While technology and design improvements have allowed the manufacture of ultrasonic imaging apparatuses that are portable, there are still a number of problems that these miniaturized apparatuses do not address. For instance, although smaller than their conventional ultrasonic imaging apparatus predecessors, prior art ultrasonic imaging apparatuses still require the doctor or examining clinician to look away from the patient in order to visualize or focus on the image displayed on the display screen. This inability to maintain the same visual axis forces the doctor to have to repeatedly look back and forth between the patient and the display. Such a process can be both time consuming and disorienting.

Having to look away from the patient in order to visualize the display screen is not only time consuming cumbersome, it can also contribute to patient injury. This is particularly true when the imaging apparatus is used to facilitate the placement and/or insertion of a hypodermic needle or catheter. In addition to being used as a diagnostic tool, ultrasonic imaging apparatuses are used to perform treatment procedures such as, for example, drawing fluids from the abdomen (paracentesis) and chest cavity (thoracentesis), and drawing blood from the space surrounding the heart (pericardiocentesis). They are also used to assist in the placement and insertion of catheters into arteries or veins (central venous catheterization). Consequently, having to look back and forth between the needle or catheter and the display screen not only makes treatment more difficult, it can also contribute to patient injury by misplacement or improper insertion of the needle or catheter.

In addition to the problem of having to look back and forth between the needle or catheter and the display, traditional methods of ultrasound guided intravenous (IV) access require the operator to continually adjust the orientation of the transducer scan head so that the sound beam emitted from the scan head crosses the tip of the needle or catheter. It is important that the operator have a clear view of the needle or catheter tip, so that the needle or catheter can be inserted properly and without injury into the intended internal target body structure. If only the shaft 200 of the needle or catheter passes through the sound beam, as illustrated in FIG. 2, the operator is only able to see the shaft 200 on the display screen, and the actual location of the tip 202 remains unknown. Not knowing where the needle or catheter tip 202 is can be potentially dangerous. If the tip 202 is inserted too deep or off to the side of the intended internal target body structure 204, an inadvertent puncture of an unintended body structure (e.g., an artery or lung) can result. The only way in which this problem could be avoided using conventional prior art ultrasound guided IV access systems would be to insert the needle or catheter at a near 90° angle (i.e., almost perpendicular to the patient's skin). Then the operator would be able to see an image of the tip of the needle or catheter on the display. Unfortunately, as can be seen in FIG. 2, a near 90° angle is not possible due to the presence of the ultrasound probe 100, which obstructs the ability to insert the needle or catheter at such an angle.

There is at least one known prior art attempt that addresses the problem of guiding and inserting a needle into a patient's body using ultrasonic imaging techniques. This attempt is disclosed in the Imran et al. patent referred to above. Beginning at line 45 of column 9 of the Imran et al. patent, an ultrasound-guided probe placement apparatus is described. The probe guide consists of a body and first and second parallel-spaced-apart forwardly extending legs. Together, the body and adjoining legs form a continuous lower planar surface that is adapted to be placed in engagement with a patient's skin. The body also includes an elongated transversely extending recess that is configured to receive the scan head of the imaging apparatus housing, and to retain the housing at a fixed angle of, for example, 45° with respect to the planar surface. The recess opens through the bottom planar surface so that the scan head can come in contact with the surface of the patient's skin that overlies the internal body structure of interest in the patient's body. A carriage is slidably mounted on the legs of the body and is movable along the lengths of the legs. The carriage is in the form of a planar member, which extends across a space between the legs. Openings are provided in the planar member overlying the upper surfaces of the legs so that scaling indicia on the upper surfaces of the legs are visible. A probe guide member is formed integral with the planar member and extends upwardly and forwardly therefrom at fixed angle of, for example, 45°. The probe guide member is provided with a longitudinally extending recess, which is semicircular in cross section, and sized to receive hypodermic needles of various sizes.

In preparation of inserting a needle into a patient's body using the probe guide apparatus of Imran et al., an operator first moves the probe guide over the skin of the patient's body until the desired image is lined up and centered with a line formed on the screen of the probe apparatus. Then, the carriage of the probe guide is moved so that its indicator is at the same numerical position as the numerical value of scaling indicia along a side of the display of the probe apparatus housing. Once the indicia are matched, a needle can then be guided into the recess of the probe guide member.

While the probe guide and carriage mechanism disclosed in Imran et al. does provide limited assistance to an operator guiding a needle to an intended internal body structure, there are a number of drawbacks with this approach. First, the indicators and scaling indicia of the probe guide and probe apparatus must be carefully calibrated to ensure accurate correspondence between the target image and the actual point at which the needle is inserted into the patient. Second, both the scan head and needle recesses appear to be set to fixed angles during the time the needle is being inserted into the patient's body. This lack of flexibility in modifying the scan head angle and/or the needle access angle during insertion of the is problematic since it may not allow the needle to be inserted to the proper depth of the intended internal target body structure and/or may cause inadvertent punctures of non-target body structures. Third, because the probe guide and carriage assembly are physically separable from the probe apparatus, i.e., is not always used with the probe apparatus, there is a possibility that the probe guide and carriage assembly could be misplaced or otherwise not available when its assistance is needed. Fourth, the probe guide and carriage assembly add to the cost and complexity of the overall design. Finally, similar to the problem with other prior art ultrasonic imaging systems, and even though a probe guide is provided, the operator is still unable to simultaneously visualize or focus on the image being displayed on the display screen while at the same time viewing insertion of the needle.

It would be desirable, therefore, to have an ultrasonic imaging apparatus and/or system which can be: easily transported; does not require an operator to look away from the patient when treating the patient with a treatment needle or catheter; provides safe and unobstructed needle or catheter access and entry; and/or which may be formed as a single unitary device.

### BRIEF SUMMARY OF THE INVENTION

A hand-held ultrasonic imaging probe and system and methods of their operation are disclosed. According to one aspect of the invention, an ultrasonic imaging probe apparatus comprises a housing containing a display and a transducer. The display is configured within a first surface of the housing, and the transducer is configured within an opposing second surface of the housing. When the second surface of the housing is laid on a surface of a patient's skin, the transducer faces the patient's skin and the display faces an opposite direction. The transducer and the display are relatively configured so that an operator may look directly down at the patient and, at the same time, visualize images of the patient's internal body structures on the display. Images are generated by sending electrical signals from the transducer, via a cable, to an external processing circuit housed within a commercially available ultrasonic imaging system. Digitized image frames generated by the external system are sent back to the probe apparatus, via the cable, for display on the integrated display.

The probe apparatus may be used to facilitate the placement and insertion of a needle or catheter into the patient. Accordingly to this aspect of the invention, images displayed on the screen (e.g., the tip of the needle or catheter and the intended internal target body structure) and the actual needle or catheter itself are maintained within the same visual axis, so there is no need to look away from the needle or catheter in order to visualize images on the display.

According to another aspect of the invention, a unitary ultrasonic imaging system is disclosed. The unitary ultrasonic imaging apparatus is similar to the probe apparatus, but also includes transducer control and signal processing circuitry. The transducer control and signal processing circuitry is used to generate beam forming signals for the transducer and to receive electrical signals characterizing sound wave reflected from the patient's internal body structures. It is also used to process and generate digital image frames for display on the integrated display. Hence, this aspect of the invention provides a completely integrated ultrasonic imaging solution, without the need for cables or processing contributions provided by an external ultrasonic imaging system. The display and transducer are configured similar to the probe apparatus summarized above, which allows an operator to look directly down at the patient and, at the same time, visualize images of the patient's internal body structures on the integrated display.

Further features and advantages of the present invention, as well as the structure and operation of various embodiments of the present invention, are described in detail below with respect to accompanying drawings, in which like reference numbers are used to indicate identical or functionally similar elements.

### BRIEF DESCRIPTION OF THE DRAWINGS

FIG. 1 is a drawing illustrating the use of a typical prior art ultrasonic imaging system;

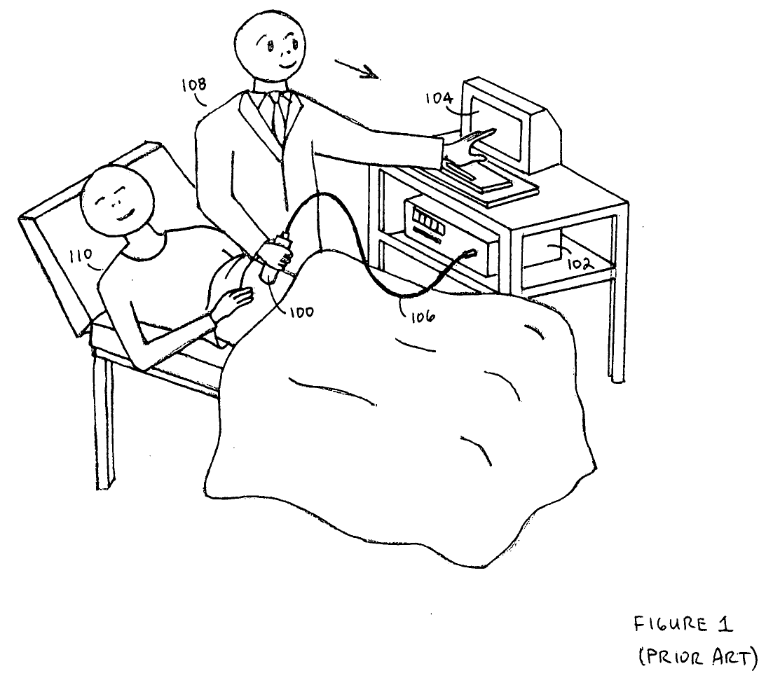

FIG. 2 is a drawing illustrating how a conventional ultrasound probe obstructs the ability of a probe operator to insert a needle or catheter at a near 90° angle relative to the surface of a patient's skin;

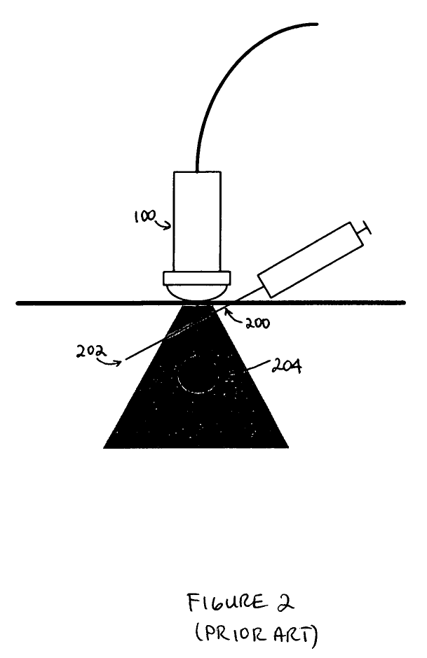

FIG. 3A is a front-view drawing of an exemplary ultrasonic imaging probe 30, according to embodiment of the present invention;

FIG. 3B is a back-view drawing of the ultrasonic imaging probe shown in FIG. 3A;

FIG. 4 is a drawing illustrating use of the ultrasonic imaging probe in FIG. 3 to guide the insertion of a needle or catheter toward and into an intended internal body structure of a patient, according to an embodiment of the present invention;

FIG. 5 is a is a flow chart illustrating an exemplary method of performing treatment of a patient using the ultrasonic imaging probe in FIG. 3, according to an embodiment of the present invention;

FIG. 6 is a drawing illustrating how, using the ultrasonic imaging probe in FIG. 3, an operator is able to insert and guide a needle or catheter toward an intended internal body structure of a patient at an angle that is substantially perpendicular to the surface of the patient's skin;

FIG. 7 is a front-view drawing of an exemplary unitary ultrasonic imaging system, according to an embodiment of the present invention;

FIG. 8 is a block diagram of an exemplary transducer control and signal processing circuit, which can be used to implement the transducer control and signal processing circuitry of the unitary ultrasonic imaging system in FIG. 7; and

FIG. 9 is a drawing illustrating use of the unitary ultrasonic imaging system in FIG. 7 to guide the insertion of a needle or catheter toward and into an intended internal body structure of a patient, according to an embodiment of the present invention.

### DETAILED DESCRIPTION OF THE INVENTION

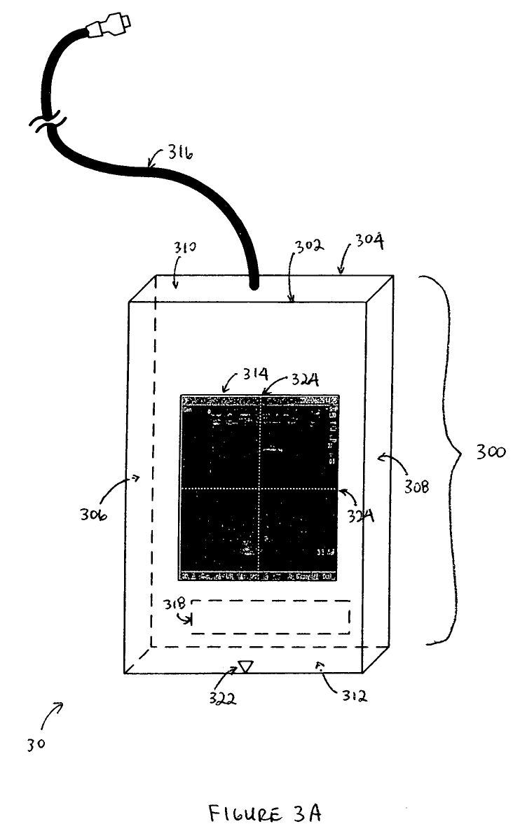

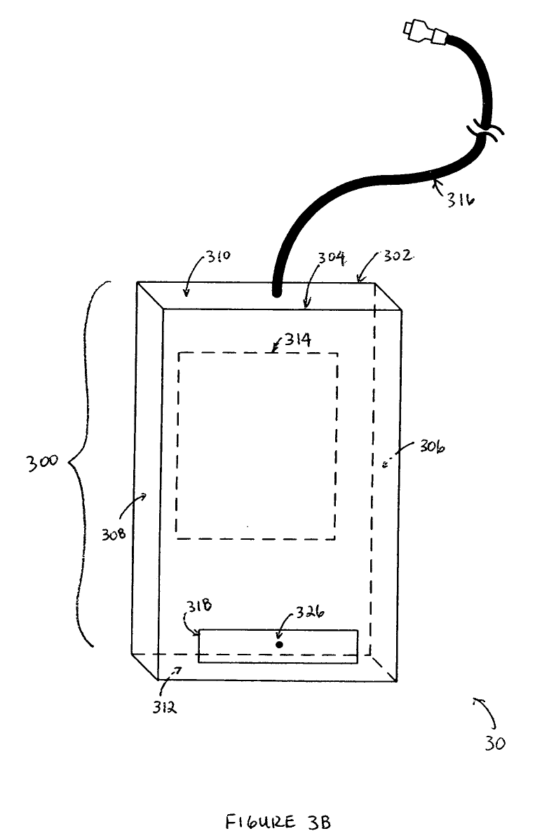

Referring first to FIG. 3A, there is shown a front view of an exemplary ultrasonic imaging probe 30, according to embodiment of the present invention. The probe 30 comprises a housing 300 made from plastic or other suitable material. The housing 300 has a front wall 302 and an opposing back wall 304. The front and back walls 302, 304 are bounded by left 306, right 308, top 310 and bottom 312 walls. A display (e.g., a liquid crystal display (LCD)) 314 is configured within the front wall 302 of the housing 300 so that the display 314 is visible to an operator when the back wall 304 of the housing 300 is placed against the surface of a patient's skin. A proximal end of a probe cable 316 is fitted through an opening in the top wall 310, and is electrically connected to the display 314 and a transducer 318, which as explained below is configured within the back wall 304 of the housing 300. A distal end of the probe cable 316 is fitted with a connector, which can mate with a corresponding probe cable connector of a commercially available ultrasonic imaging apparatus.

FIG. 3B shows a back view of the ultrasonic imaging probe 30. As shown, a piezoelectric transducer 318 is configured within the back wall 304 of the housing 300, and is adapted so that it can be easily placed against the surface of a patient's skin. The transducer 318 is comprised of a plurality of piezoelectric transducer elements formed in an array. Depending on the application, the array may be configured in the form of a linear array, a phased array, a curved array, etc., as is well known in the ultrasonographic arts. Although not required, the bottom edge 320 of the transducer 318 may be configured so that it either abuts or is in close proximity to the plane defining the bottom wall 312 of the housing 300. Further, whereas the display 314 and transducer 318 are shown to only partially overlap, in an alternative embodiment the display 314 and transducer 318 may be configured so that the display 314 completely overlaps the transducer 318.

As shown in FIG. 3A, the housing 300 may also include an intravenous (IV) access marker 322 centered along the bottom edge of the front wall 302. The relative positions of the display 314 and transducer 318 are configured so that the IV access marker 322 either corresponds to the center of the display screen 314 or corresponds with some other predetermined point on the display 314. The display 314 may also include center line markers 324 defining the center of the display 314 and/or other markers to define other predetermined points on the display 314. As shown in FIG. 3B, the transducer 318 may be also provided with a dot or line 326, an image of which appears on the display 314 during scanning. The image of the dot or line 326 assists the operator in moving and positioning the probe 30 in the proper direction. One or more of these features also helps to ensure that a needle or catheter is properly directed toward the intended internal target body structure, when an operator directs the needle or catheter into the patient.

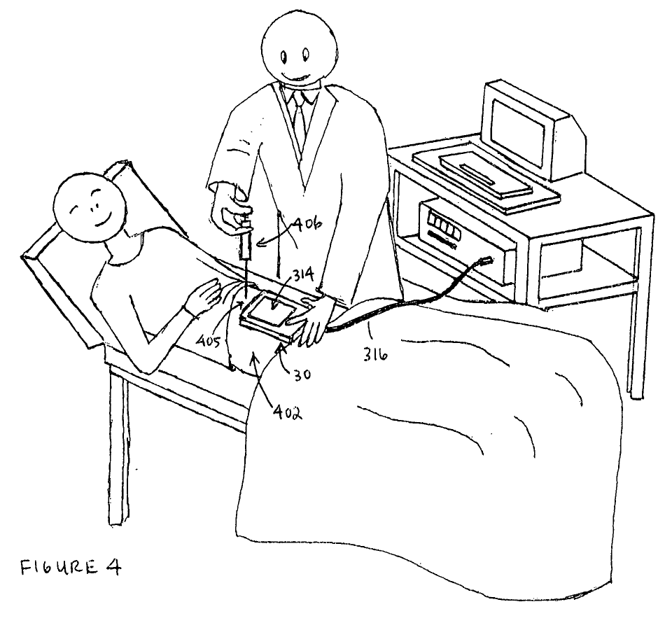

FIG. 4 is a diagram illustrating use of the ultrasonic imaging probe 30 to guide the insertion of a needle or catheter 406 toward and into an intended internal body structure (e.g. a vein) of a patient. The probe 30 is positioned so that the back wall 304 of the housing 300 is positioned flat against the patient's skin 402. When in this position, the transducer 318 is also placed flat against the patient's skin 402, so that it can direct sound pulses toward the intended internal body structure. Reflected sound waves from the intended internal body structure impinge on the transducer 318, which causes the transducer to vibrate and generate electrical signals. The electrical signals are transmitted to a commercially available ultrasonic imaging system via the probe cable 316. Processing circuitry in the external ultrasonic imaging system digitizes and processes the electrical signals into digital display signals. These display signals are transmitted back to the probe 30, where they are available for display on the display 314 of the probe 30.

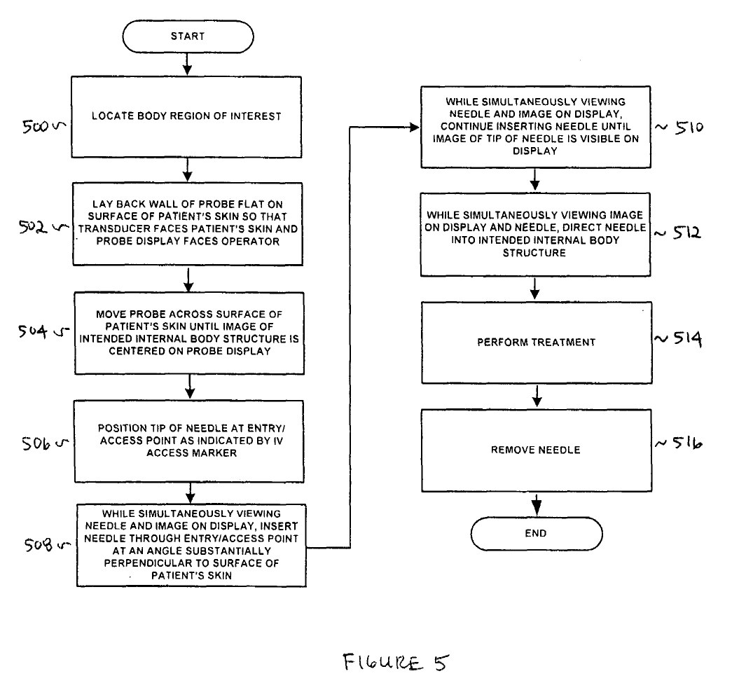

FIG. 5 is a flow chart illustrating an exemplary method of performing treatment of a patient using the ultrasonic imaging probe 30 discussed above. According to a first step 500, the probe operator locates a body region of interest of the patient receiving treatment (e.g., an area over the patient's chest cavity or abdomen).

Next, at step 502, the probe operator lays the back wall of the probe 30 flat on the surface of the patient's skin in the body region of interest, so that the transducer 318 faces toward the patient's skin, and so that the probe display 314 faces toward the operator and is within the field of vision of the operator.

At step 504, the probe operator moves the probe 30 across the surface of the patient's skin to capture an image of the intended internal body structure 404. As this step 504 is performed, the transducer 318 sends electrical signals characterizing the reflected and received sound waves to processing circuitry in the external ultrasonic imaging apparatus. The processing circuitry in the external ultrasonic imaging apparatus generates a succession of image frames, which are transmitted back to the probe 30 and displayed on the probe display 314. Once the operator views an image frame showing the intended internal body structure 404, the operator makes a minor adjustment of the placement of the probe 30, so that intended internal body structure 404 becomes centered on a subsequent image frame displayed on the probe display 314. The probe 30 is then held in a stationary position in preparation of the next step.

At step 506, the operator positions the tip of a treatment needle or catheter 406 proximate an access/entry point 405 (see FIG. 4) of the patient, as indicated by the IV access marker 322. Because the display 314 is in close proximity to the access/entry point 405, both the display 314 and the needle or catheter 406 are within the same visual axis of the operator. This configuration allows the operator to visualize images of the intended internal body structure 404 on the display 314 while at the same time viewing the needle or catheter 406 and/or the access/entry point 405. The ability to simultaneously visualize both the images on the display and the actual access/entry point 405 allows for safe insertion of the needle or catheter 406, since the operator does not have to look away from either the needle or catheter 406 or the access/entry point 405 in order to view the image on the display 314.

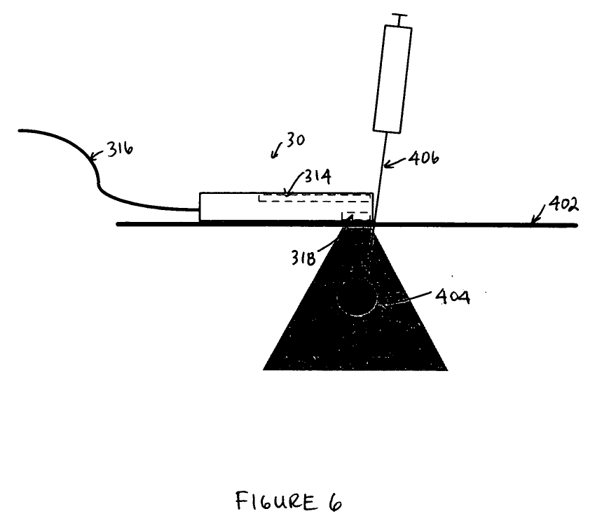

Next, at step 508, the operator inserts the needle or catheter 406 through the access/entry point 405 and into the patient's body, while simultaneously viewing the image on the display 314 and the needle or catheter 406. The configuration of the housing 300, transducer 318 and display 314 of the probe 30 allows the operator to position and guide the needle or catheter 406 toward the intended body structure 404 without having to look away from either the needle or catheter 406 or the access/entry point 405. Further, and as illustrated in FIG. 6, because the probe 30 is laid flat against the patient's skin, the probe operator is able to insert and guide the needle or catheter 406 toward the intended internal body structure 404 at an angle that is substantially perpendicular to the surface of the patient's skin.

At step 510, the operator continues to insert the needle or catheter 406 into the patient and toward the intended internal body structure 404 while simultaneously viewing the image on the display 314 and the needle or catheter 406. Because the needle or catheter 406 can be inserted at a near 90° angle relative to the patient's skin, and within the sound beam being projected by the transducer array 318, eventually an image frame including an image of the tip of the needle or catheter 406 will be displayed on the display 314. The ability to view images of both the intended internal body structure 404 and the tip of the needle or catheter 406 on the display 314, while not having to look away from the needle or catheter 406 and/or access/entry point 405 prevents against inadvertent punctures of unintended body structures.

At step 512, while simultaneously viewing the image of the internal body structure 404 on the display 314, the image of the tip of the needle or catheter 406 on the display, and the actual needle or catheter 406 itself, the operator directs the tip of the needle or catheter 406 into the intended internal body structure 404. Once the tip of the needle or catheter 406 is properly inserted, at step 514 the operator performs the necessary treatment (e.g., aspirating blood or body fluid, injecting a drug, configuring an IV, etc.) Finally, once the treatment in step 514 is completed, at step 516 the needle or catheter 406 is removed from the patient and the method is complete.

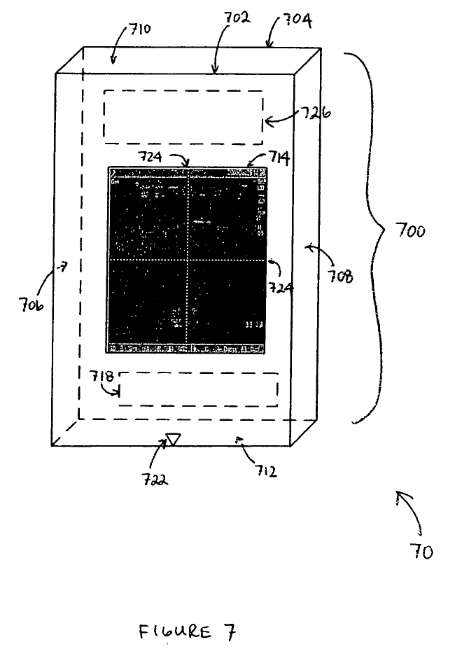

Referring now to FIG. 7, there is shown a diagram of an exemplary unitary ultrasonic imaging system 70, according to another embodiment of the present invention. The unitary ultrasonic imaging system 70 is similar to the ultrasonic imaging probe 30 described above, and, as explained in more detail below, also includes circuitry for controlling both the beam forming operations of the transducer and processing the electrical signals received from the transducer. The unitary ultrasonic imaging system 70 comprises a housing 700 made from plastic or other suitable material. The housing 700 has a front wall 702 and an opposing back wall 704. The front and back walls 702, 704 are bounded by left 706, right 708, top 710 and bottom 712 walls. Also, similar to the probe 30 described above, the unitary ultrasonic imaging system 70 includes a display (e.g., an (LCD)) 714 and a transducer 718. The display 714 is configured within the front wall 702 of the housing 700 so that the image frames displayed on the display 714 are visible to an operator when the back wall 704 of the housing 700 is placed against the surface of a patient's skin. The transducer 718 is configured within the back wall 704 of the housing 700, and is adapted so that it can be easily placed against the surface of the patient's skin. The transducer 718 is comprised of a plurality of piezoelectric transducer elements formed in an array. Depending on the application, the transducer array 718 may be configured in the form of a linear array, a phased array, a curved array, etc., as is well known in the ultrasonographic arts. Although not required, the bottom edge 720 of the transducer 718 may be configured so that it either abuts or is in close proximity to the plane defining the bottom wall 712 of the housing 700. Further, whereas the display 714 and transducer 718 are shown to only partially overlap, in an alternative embodiment the display 714 and transducer 718 may be configured so that the display 714 completely overlaps the transducer 718.

Similar to the probe 30 in FIG. 3 above, the housing 700 of the unitary ultrasonic imaging system 70 may also include an IV access marker 722 centered along the bottom edge of the front wall 702 of the housing 700. The relative positions of the display 714 and transducer 718 are configured so that the IV access marker 722 either corresponds to the center of the display screen 714 or corresponds with some other predetermined point on the display 714. The display 714 may also include one or more center line markers 724 defining the center of the display 714 and/or other markers defining other predetermined points on the display 714. Similar to the back view of the probe 30 in FIG. 3B, the transducer 718 of the unitary ultrasonic imaging system 70 may also include a dot or line, an image of which appears on the display 714 during scanning. The image of the dot or line assists the operator in moving and positioning the unitary system 70 in the proper direction. One or more of these features also helps to ensure that a needle or catheter is properly directed toward the intended internal target body structure, when an operator directs the needle or catheter into the patient.

Also included within the housing of the unitary ultrasonic imaging system 70 is transducer control and signal processing circuitry 726, which is electrically coupled to the transducer array 718 and the display 714. The transducer control and signal processing circuitry 726 is adapted to receive electrical signals received from the transducer array 718, and to generate beam forming signals for the transducer array 718. The transducer control and signal processing circuitry 726 is also adapted to generate and process digital display signals from the electrical signals received from the transducer 718 for displaying images on the display 714. Further details concerning the operation and configuration of the circuitry 726 are provided below.

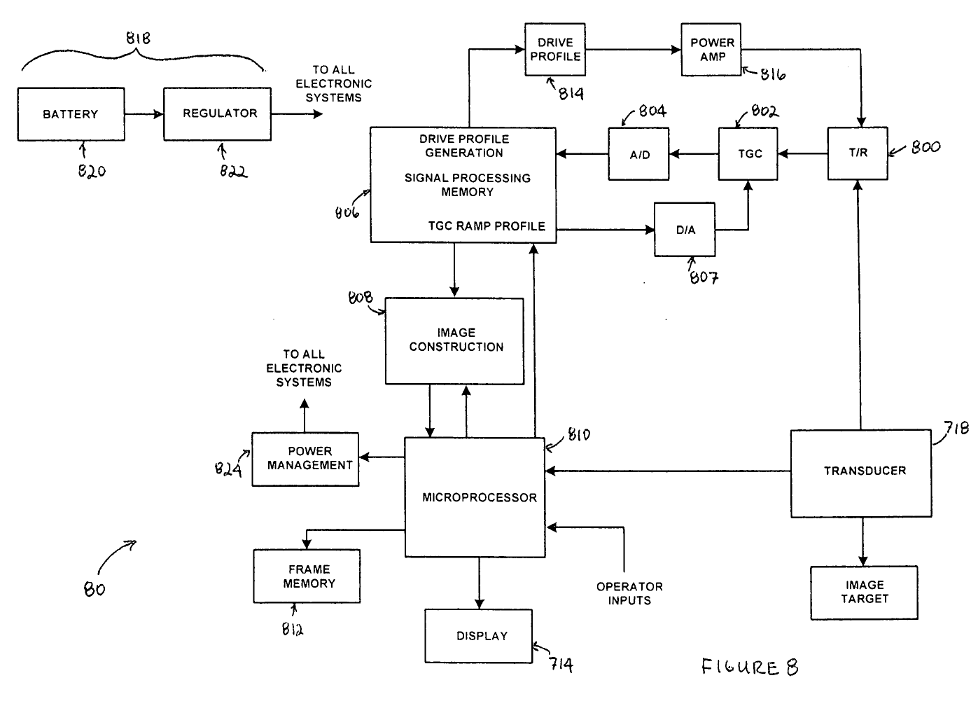

FIG. 8 shows an exemplary transducer control and signal processing circuit 80, which can be used to implement the circuitry 726 in FIG. 7. The circuit 80 is similar to that disclosed in U.S. Pat. No. 6,569,102 to Imran et al., which is incorporated herein by reference. Further details of the various components and their functions and operation can be found by reference to that patent. U.S. Pat. No. 5,817,024 to Ogle et al., which is also incorporated by reference, discloses an alternative circuit which can be used to implement the circuitry 726 in FIG. 7.

As shown in FIG. 8, the transducer array 718 is connected to a transmit/receive (T/R) switch 800 by a number of channels corresponding to the plurality of transducer elements of the transducer array 718. The T/R switch 800 is operable to transmit signals to the transducer array 718 in a transmit mode, and to receive signals from the transducer array 718 in a receive mode. The T/R switch 800 may be implemented in various ways. According to one implementation, a plurality of diodes is used. The diodes are biased on and off to perform switching between transmit and receive modes of the transducer array 718.

In the receive mode, the transducer array 718 converts sound waves reflected from the internal body structures of a patient into electrical signals. These electrical signals are switched by the T/R switch 800 to the input of a time-gain-compensation (TGC) amplifier 802. The TGC amplifier 802 operates to compensate for scattering and attenuation of ultrasonic energy of sound waves as they penetrate deeper into the patient's tissue. Compensation is achieved by amplifying the electrical signals according to the amount of time it takes for the corresponding sound waves to reflect from the underlying tissue. The TGC amplifier 802 amplifies the received electrical signals accordingly and couples the resulting amplified signals the input of an analog-to-digital (A/D) converter 804. The A/D converter 804 digitizes the amplified signals into raw digital data, which is stored in signal processing memory of a field programmable gate array (FPGA) 806. The signal processing memory of the FPGA 806 serves as a data buffer that stores the raw digital data until it is need for image construction. The FPGA 806 is also configured to generate digital time-gain-compensation ramp profile signals, which are converted to analog signals by a digital-to-analog (D/A) converter 807 and supplied to a control input of the TGC amplifier 802.

Image construction is performed by a digital signal processing (DSP) circuit or chip 808, which is under the control of a microprocessor 810. The DSP circuit 808 is operable to analyze the amplitudes of the received signals to provide a gray scale. Zoom function, Doppler processing and color flow can also be implemented by the DSP circuit 808.

The microprocessor 810 also performs various other tasks including, for example, supplying image frames generated by the DSP circuit 808 to the display 714, and averaging the raw digital data and/or image frames following image construction to smooth transitions between image frames. User interface inputs to the microprocessor 810 may also be included for controlling and/or accessing image or post frame averaging, ON/OFF, TGC, zoom and Doppler functions. Finally, a frame memory 812 may also be coupled to the microprocessor 810 to store a plurality of image frames, which may then be subsequently recalled.

In the transmit mode, a drive profile portion of the FPGA 806 operates to generate a drive profile. While shown as forming a portion of the FPGA 806, the drive profile portion may be implemented as a separate component. The drive profile may be in the form of a single excitation gated burst of cycles of (e.g., 3 to 5 cycles of the operating frequency of the transducer array 718) to a drive profile circuit 814. The drive profile circuit 814 serves as a buffer, which feeds the drive profile to a power amplifier (PA) 816. The PA 816 amplifies the gated burst of cycles and supplies the amplified gated burst of cycles to the T/R switch 800, and then to the transducer array 718, thereby providing corresponding excitations for the transducer array 718. The transducer array 718 responds to the excitations by generating ultrasonic pulses, which are directed towards the intended internal body structure 404 of the patient.

A power supply 818, comprising a battery 820 and a regulator 822, provides a regulated power supply to all of the electrical systems of the transducer control and signal processing circuit 80. The power supply 818 may also include a power management circuit 824 that operates to control power supplied by the battery 820 so that power use is conserved. The microprocessor 810, acting through the power management circuit 824, determines which devices of the circuit 80 have performed their respective functions or are not being utilized and places them in a power down or sleep mode until the devices are once again in need of power.

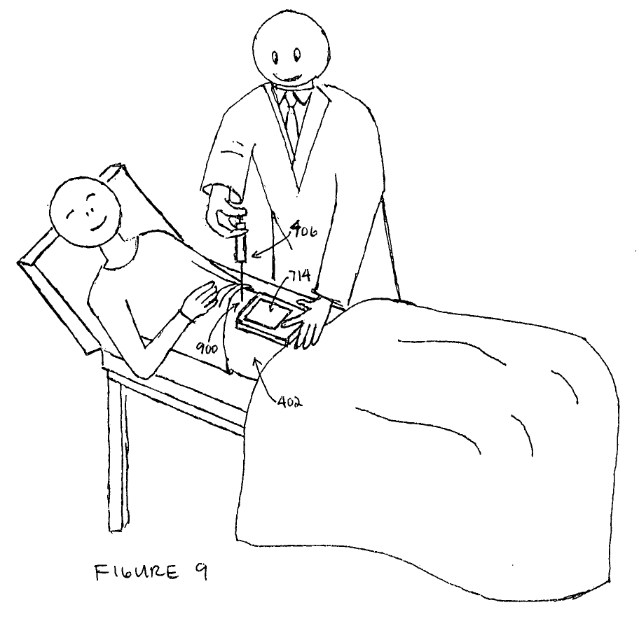

FIG. 9 is a diagram illustrating use of the unitary ultrasonic imaging system 70 to guide the insertion of a needle or catheter 406 toward and into an intended internal body structure. Similar to the probe 30 described above, because the display 714 is in close proximity to the access/entry point 900, both the display 714 and the needle or catheter 406 are within the same visual axis of the operator. As discussed above, this configuration allows an operator to visualize images of the intended internal body structure on the display 714 while at the same time viewing the needle or catheter 406 and/or the access/entry point 900. The ability to simultaneously visualize both the images on the display and the actual access/entry point 900 allows for safe insertion of the needle or catheter 406, since the operator does not have to look away from either the needle or catheter 406 or the access/entry point 900 in order to view the image on the display 714. Further, because the needle or catheter 406 can be inserted at a near 90° angle relative to the patient's skin, and within the sound beam being projected by the transducer array 718, images of both the intended internal body structure and the tip of the needle or catheter on the display 714 can be viewed while not having to look away from the needle or catheter 406 and/or access/entry point 900.

The method of performing treatment using the unitary ultrasonic imaging system 70 is similar to the method of using the ultrasonic imaging probe 30 discussed above (see FIG. 5 and corresponding description), except that the transducer control and signal processing are performed by the transducer control and signal processing circuit 80 within the housing 700 of the system 70, rather than by an external ultrasonic imaging system.

Although the present invention has been described with reference to specific embodiments thereof, these embodiments are merely illustrative, and not restrictive, of the present invention. For example, although the ultrasonic imaging probe 30 and the unitary ultrasonic imaging system 70 have been described as being used in the context of ultrasound guided IV access, both the probe 30 and unitary system may also be used to perform non-procedural diagnostic procedures (e.g., diagnostic examinations). Additionally, various modifications or changes to the specifically disclosed exemplary embodiments will be suggested to persons skilled in the art and are to be included within the spirit and purview of this application and scope of the appended claims.

What is claimed is:

1. An ultrasonic imaging apparatus, comprising:
a housing having a first surface and a second surface opposing said first surface;
a display configured within the first surface of said housing; and
a transducer configured within the second surface of said housing.

2. The ultrasonic imaging apparatus of claim 1 wherein a primary surface of said display completely overlaps a primary surface of said transducer, when said first surface is viewed along a visual axis that is perpendicular to the primary surface of said display.

3. The ultrasonic imaging apparatus of claim 1 wherein a primary surface of said display partially overlaps a primary surface of said transducer, when said first surface is viewed along a visual axis that is perpendicular to the primary surface of said display.

4. The ultrasonic imaging apparatus of claim 1 wherein said transducer has an edge that is in close proximity to a surface joining said first and second surfaces of said housing.

5. The ultrasonic imaging apparatus of claim 1 wherein the relative configuration of said display and said transducer is such that an operator may simultaneously visualize both a body region of interest of a patient and an image of an internal body structure of the patient on said display.

6. The ultrasonic imaging apparatus of claim 1 wherein said housing is designed so that when a primary surface of said transducer is positioned over a surface of a patient's skin, an operator may insert a needle or catheter through the surface of the skin at an angle that is approximately ninety degrees relative to a primary surface of said display.

7. The ultrasonic imaging apparatus of claim 1 wherein an operator can visualize an image of a tip of a needle or catheter being inserted through a surface of a patient's skin on said display and an image of an internal target body structure on said display without having to vary a visual axis used for visualizing said needle or catheter being inserted through the surface of the patient's skin.

8. The ultrasonic imaging apparatus of claim 1 wherein an operator can visualize an image of a tip of a needle or catheter being inserted through a surface of a patient's skin on said display and an image of an internal target body structure on said display while simultaneously visualizing said needle or catheter being inserted through the surface of the patient's skin.

9. The ultrasonic imaging apparatus of claim 1, further comprising an electrical cable having a first end electrically coupled to said display and said processor and a second end adapted to be coupled to an external processing circuit.

10. The ultrasonic imaging apparatus of claim 1, further comprising a transducer control and processing circuit configured within said housing and electrically coupled to said transducer and said display.

11. A method of treating a patient using an ultrasonic imaging apparatus, wherein said apparatus includes a housing having a first surface and an opposing second surface, a display configured within the first surface, and a transducer configured within the second surface, said method comprising:
positioning the second surface of said housing of said ultrasonic imaging apparatus on a surface of a patient's skin, so that said transducer faces said surface of the patient's skin;
repositioning the apparatus until an image of an internal target body structure appears on said display;
inserting a treatment needle or catheter through an access/entry point on the surface of the patient's skin; and
while simultaneously visualizing images on said display and said needle or catheter, directing a tip of said needle or catheter into said internal target body structure.

12. The method of claim 11 wherein inserting a treatment needle or catheter through an access/entry point on the surface of the patient's skin is performed while simultaneously visualizing both said image of said internal body structure on said display and said needle or catheter.

13. The method of claim 11 wherein said treatment needle or catheter is inserted through said access/entry point at an angle that is substantially perpendicular to the surface of said patient's skin.

14. The method of claim 11 wherein simultaneously visualizing images on said display and said needle or catheter comprises visualizing an image of the tip of said needle or catheter being inserted through a surface of a patient's skin on said display and an image of an internal target body structure on said display while simultaneously visualizing said needle or catheter being inserted through the surface of the patient's skin.

15. The method of claim 11 wherein simultaneously visualizing images on said display and said needle or catheter comprises visualizing an image of the tip of the needle or catheter being inserted through a surface of a patient's skin on said display and an image of an internal target body structure on said display without having to vary a visual axis used for visualizing said needle or catheter being inserted through the surface of the patient's skin.

16. An ultrasonic imaging apparatus, comprising:
a housing having a front wall and a back wall;
a transducer configured within the back wall, said transducer adapted so that it is positioned against a surface of a patient's skin when said back wall is placed on the patient's skin; and
a display configured within the front wall, said display adapted so that it is visible to an operator when said back wall is placed on the patient's skin.

17. The ultrasonic imaging apparatus of claim 16 wherein said transducer and said housing are configured so that their major surfaces are substantially parallel.

18. The ultrasonic imaging apparatus of claim 16, further comprising a processing circuit coupled to said transducer and said display, said processing circuit operable to digitize analog signals received from said transducer and generate digital display signals for forming images on said display.

19. The ultrasonic imaging apparatus of claim 16 wherein said transducer is configured within said back wall so that at least one of its edges is in close proximity to a wall joining the front and back walls.
